(19) United States
(12) Patent Application Publication
(10) Pub. No.: US 2016/0045496 A1
(43) Pub. Date: Feb. 18, 2016

(54) USE OF (RS)-S-CYCLOPROPYL-S-(4--5-(TRIFLUOROMETHYL)PYRIMIDIN-2-YL]AMINO}PHENYL)SULFOXIMIDE FOR TREATMENT OF SPECIFIC TUMOURS

(71) Applicant: BAYER PHARMA AKTIENGESELLSCHAFT, Berlin (DE)

(72) Inventors: Gerhard SIEMEISTER, Berlin (DE); Vincent RIBRAG, Paris (FR); Valérie CAMARA-CLAYETTE, Versailles (FR)

(21) Appl. No.: 14/773,656

(22) PCT Filed: Feb. 28, 2014
(86) PCT No.: PCT/EP2014/053975

§ 371 Date: Sep. 8, 2015

(30) Foreign Application Priority Data
Mar. 7, 2013 (DE) 10 2013 203 913.1

(51) Int. Cl. A61K 31/505

(52) U.S. Cl. CPC A61K 31/505

(57) ABSTRACT

The invention relates to the use of (R)-S-cyclopropyl-S-(4-{[4-{[(1R, 2R)-2-hydroxy-1-methylpropyl]oxy}-5-(trifluoromethyl)pyrimidin-2-yl]amino}phenyl)sulphoximide and/or (S)-S-cyclopropyl-S-(4-{[4-{[(1R, 2R)-2-hydroxy-1-methylpropyl]oxy}-5-(trifluoromethyl)pyrimidin-2-yl]amino}phenyl)sulphoximide for treating specific tumours.

The present invention relates to the use of (RS)-S-cyclopropyl-S-(4-{[4-{[(1R, 2R)-2-hydroxy-1-methylpropy]oxy}-5-(trifluoromethyl)pyrimidin-2-yl]amino}phenyl)sulphoximide, more particularly (R)-S-cyclopropyl-S-(4-{[4-{[(1R, 2R)-2-hydroxy-1-methylpropyl]oxy}-5-(trifluoromethyl)pyrimidin-2-yl]amino}phenyl)sulphoximide, for treating specific tumours.

Cyclin-dependent kinases (CDK) are a family of enzymes which plays an important role in the regulation of the cell cycle and thus represents an especially interesting target for the development of small inhibitory molecules. Selective inhibitors of CDKs can be used to treat cancer or other diseases caused by faults in cell proliferation.

Pyrimidines and analogues have already been described as active ingredients, for example the 2-anilinopyrimidines as fungicides (DE 4029650) or substituted pyrimidine derivatives for treating neurological or neurodegenerative disorders (WO 99/19305). A very wide variety of different pyrimidine derivatives have been described as CDK inhibitors, for example 2-amino-4-substituted pyrimidines (WO 01/14375), purines (WO 99/02162), 5-cyanopyrimidines (WO 02/04429), anilinopyrimidines (WO 00/12486) and 2-hydroxy-3-N,N-dimethylaminopropoxypyrimidines (WO 00/39101).

More particularly, WO 02/096888 and WO 03/076437 disclosed pyrimidine derivatives which have inhibitory actions with regard to CDKs.

Examples of sulphoximine active ingredients are sulphonimidoyl-modified triazoles as fungicides (H. Kawanishi, H. Morimoto, T. Nakano, T. Watanabe, K. Oda, K. Tsujihara, Heterocycles 1998, 49, 181) or arylalkylsulphoximines as herbicides and pesticides (Shell International Research, Ger. P. 2 129 678).

WO 2005/037800 discloses open sulphoximine-substituted anilinopyrimidine derivatives as inhibitors of cyclin-dependent kinases. Examples given are structures which, in the 5-position of the pyrimidine, are either unsubstituted or substituted by halogen, more particularly by bromine None of the specifically disclosed structures has a 5-trifluoromethyl substituent.

The novel pan-CDK inhibitors and methods for the preparation thereof are described in the PCT application PCT/EP2009/007247, the disclosure of which is referred to in the present application and which is incorporated into this application by reference. (RS)-S-(4-{[4-{[(1R, 2R)-2-Hydroxy-1-methylpropyl]oxy}-5-(trifluoromethyl)pyrimidin-2-yl]amino}phenyl)-S-methylsulphoximide is exemplary compound 1.

The use of a group of pan-CDK inhibitors in various tumour diseases is the subject matter of PCT/EP2011/054733. (RS)-S-Cyclopropyl-S-(4-{[4-{[(1R, 2R)-2-hydroxy-1-methylpropyl]oxy}-5-(trifluoromethyl)pyrimidin-2-yl]amino}phenyl)sulphoximide is exemplary compound 1.

The combination of the aforementioned group of pan-CDK inhibitors with other tumour therapeutics in various tumour diseases is the subject matter of DE102010014427. (RS)-S-Cyclopropyl-S-(4-{[4-{[(1R, 2R)-2-hydroxy-1-methylpropyl]oxy}-5-(trifluoromethyl)pyrimidin-2-yl]amino}phenyl)sulphoximide is exemplary compound 1.

Proceeding from this prior art, it is an object of the present invention to provide compounds for patients suffering from lymphomas, more particularly diffuse large B-cell lymphomas, rhabdomyosarcomas, neuroblastomas or squamous-cell carcinomas, especially of the lungs, the head and nape or the cervix.

From the oncological efficacy of a compound in a specific indication, it is not possible to predictably infer oncological efficacy in other specific indications as well.

Tumours differ, inter alia, in their degree of differentiation, in their vascularization, in the formation of hypoxic or necrotic areas and in their metabolic adaptation. In summary, the picture is one of great heterogeneity, which can be reflected in the manner of the response to medicinal treatment.

This hetereogeneity, which is attributed not only to the tissue of origin, but also to the nature and number of accumulated genomic modifications, for example mutations and amplifications, can also be found at the tumour cell level. The strong variation of the response to oncological active ingredients even at the cellular level is, for example, impressively shown in the analysis by the US Food and Drug Administration of data from the NCI 60 panel of human tumour cell lines (Holbeck, S. L., Collins, J. M., Doroshow, J. H., Analysis of Food and Drug Administration—Approved Anticancer Agents in the NCI60 Panel of Human Tumour Cell Lines. Mol Cancer Ther; 9(5); 1451-1460, 2010).

It has now been found that the compound (RS)-S-cyclopropyl-S-(4-{[4-{[(1R, 2R)-2-hydroxy-1-methylpropyl]oxyl}-5-(trifluoromethyl)pyrimidin-2-yl]amino}phenyl)sulphoximide (compound A), more particularly (R)-S-cyclopropyl-S-(4-{[4-{[(1R, 2R)-2-hydroxy-1-methylpropyl]oxyl}-5-(trifluoromethyl)pyrimidin-2-yl]amino}phenyl)sulphoximide (compound A′), acts in specific tumour types which had previously not yet been contemplated, viz. in lymphomas, more particularly in diffuse large B-cell lymphomas or mantle cell lymphomas, in rhabdomyosarcomas, neuroblastomas or squamous-cell carcinomas, especially of the lungs, the head and nape or the cervix.

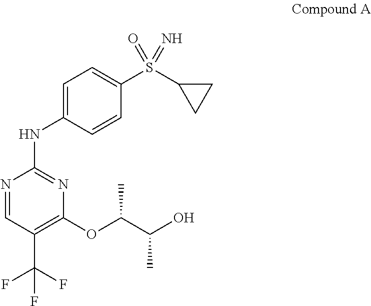 [SMILES: C[C@@H](O)[C@@H](C)Oc1nc(Nc2ccc(S(=N)(=O)C3CC3)cc2)ncc1C(F)(F)F]

(RS)-S-Cyclopropyl-S-(4-{[4-{[(1R, 2R)-2-hydroxy-1-methylpropyl]oxyl}-5-(trifluoromethyl)pyrimidin-2-yl]amino}phenyl)sulphoximide (compound A) is a selected sulphoximine-substituted anilinopyrimidine derivative which can be separated into two stereoisomers, viz.:
(R)-S-cyclopropyl-S-(4-{[4-{[(1R, 2R)-2-hydroxy-1-methylpropyl]oxy}-5-(trifluoromethyl)pyrimidin-2-yl]amino}phenyl)sulphoximide (compound A′) and
(S)-S-cyclopropyl-S-(4-{[4-{[(1R, 2R)-2-hydroxy-1-methylpropyl]oxy}-5-(trifluoromethyl)pyrimidin-2-yl]amino}phenyl)sulphoximide (compound A″).

Compound A′ is preferred and in clinical development as BAY1000394.

Where compound A is mentioned below, both the pure stereoisomers A′ and A″, and also any mixture of these two, are meant thereby.

The present application provides for the use of
(RS)-S-cyclopropyl-S-(4-{[4-{[(1R, 2R)-2-hydroxy-1-methylpropyl]oxy}-5-(trifluoromethyl)pyrimidin-2-yl]amino}phenyl)sulphoximide,
more particularly
(R)-S-cyclopropyl-S-(4-{[4-{[(1R, 2R)-2-hydroxy-1-methylpropyl]oxy}-5-(trifluoromethyl)pyrimidin-2-yl]amino}phenyl)sulphoximide,
for treating lymphomas, more particularly diffuse large B-cell lymphomas or mantle cell lymphomas, rhabdomyosarcomas, neuroblastomas or squamous-cell carcinomas, especially of the lungs, the head and nape or the cervix.

The present application further provides for the use of
(RS)-S-cyclopropyl-S-(4-{[4-{[(1R, 2R)-2-hydroxy-1-methylpropyl]oxy}-5-(trifluoromethyl)pyrimidin-2-yl]amino}phenyl)sulphoximide,
more particularly
(R)-S-cyclopropyl-S-(4-{[4-{[(1R, 2R)-2-hydroxy-1-methylpropyl]oxy}-5-(trifluoromethyl)pyrimidin-2-yl]amino}phenyl)sulphoximide,
for preparing a medicament for treating lymphomas, more particularly diffuse large B-cell lymphomas or mantle cell lymphomas, rhabdomyosarcomas, neuroblastomas or squamous-cell carcinomas, especially of the lungs, the head and nape or the cervix.

The present application further provides (RS)-S-cyclopropyl-S-(4-{[4-{[(1R, 2R)-2-hydroxy-1-methylpropyl]oxy}-5 -(trifluoromethyl)pyrimidin-2-yl]amino}phenyl) sulphoximide,
more particularly
(R)-S-cyclopropyl-S-(4-{[4-{[(1R, 2R)-2-hydroxy-1-methylpropyl]oxy}-5-(trifluoromethyl)pyrimidin-2-yl]amino}phenyl)sulphoximide,
for treating lymphomas, more particularly diffuse large B-cell lymphomas or mantle cell lymphomas, rhabdomyosarcomas, neuroblastomas or squamous-cell carcinomas, especially of the lungs, the head and nape or the cervix.

The present application further provides drugs and pharmaceutical formulations containing
(RS)-S-cyclopropyl-S-(4-{[4-{[(1R, 2R)-2-hydroxy-1-methylpropyl]oxy}-5-(trifluoromethyl)pyrimidin-2-yl]amino}phenyl)sulphoximide,
more particularly
(R)-S-cyclopropyl-S-(4-{[4-{[(1R, 2R)-2-hydroxy-1-methylpropyl]oxy}-5-(trifluoromethyl)pyrimidin-2-yl]amino}phenyl)sulphoximide,
for treating lymphomas, more particularly diffuse large B-cell lymphomas or mantle cell lymphomas, rhabdomyosarcomas, neuroblastomas or squamous-cell carcinomas, especially of the lungs, the head and nape or the cervix.

The present application further provides combinations of
(RS)-S-cyclopropyl-S-(4-{[4-{[(1R, 2R)-2-hydroxy-1-methylpropyl]oxy}-5-(trifluoromethyl)pyrimidin-2-yl]amino}phenyl)sulphoximide,
more particularly
(R)-S-cyclopropyl-S-(4-{[4-{[(1R, 2R)-2-hydroxy-1-methylpropyl]oxy}-5-(trifluoromethyl)pyrimidin-2-yl]amino}phenyl)sulphoximide,
with at least one further active ingredient for treating lymphomas, more particularly diffuse large B-cell lymphomas or mantle cell lymphomas, rhabdomyosarcomas, neuroblastomas or squamous-cell carcinomas, especially of the lungs, the head and nape or the cervix.

The use of the physiologically tolerable salts of compound A should likewise be considered to be covered by the present invention.

Physiologically safe salts of compound A encompass acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically safe salts of compound A also encompass salts of customary bases, such as, by way of example and preferably, alkali metal salts (e.g. sodium and potassium salts), alkaline earth metal salts (e.g. calcium and magnesium salts) and ammonium salts derived from ammonia or organic amines having from 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

The present invention further provides drugs containing compound A and at least one or more further active ingredients for treating lymphomas, more particularly diffuse large B-cell lymphomas or mantle cell lymphomas, rhabdomyosarcomas, neuroblastomas or squamous-cell carcinomas, especially of the lungs, the head and nape or the cervix.

Compound A according to the invention can act systemically and/or locally. To this end, it can be administered in an appropriate manner, for example orally, parenterally, pulmonarily, nasally, sublingually, lingually, buccally, rectally, dermally, transdermally, conjunctivally, optically or as an implant or stent.

For these routes of administration, compound A can be delivered in an appropriate administration form.

Suitable for oral administration are administration forms which release the compounds according to the invention in a rapid and/or modified manner and function according to the prior art and which contain the compounds according to the invention in crystalline and/or amorphized and/or dissolved form, for example tablets (non-coated or coated tablets, having, for example, enteric or slow-dissolving or insoluble coats which regulate the release of the compound according to the invention), films/lyophilisates, capsules (for example, hard or soft gelatin capsules), dragées, granules, pellets, powders, emulsions, suspensions, aerosols or solutions.

Solutions containing or consisting of solubilizers, surface-active substances and/or one or more flavourings have been found to be advantageous for compound A.

Suitable solubilizers are macrogols, more particularly macrogol 400.

Suitable surface-active substances are polysorbates, more particularly polysorbate 20.

Suitable flavourings are essential oils, more particularly menthol.

The active ingredient concentration can be from 0.1 mg/ml to 10 mg/ml, preferably from 0.2 mg/ml to 8 mg/ml, particularly preferably from 0.3 mg/ml to 6 mg/ml and extremely preferably from 0.4 mg/ml to 4 mg/ml.

The concentrations 0.2 mg/ml and 4.8 mg/ml are given as examples.

Tablets containing or consisting of fillers, disintegrants and/or one or more press additives have also been found to be advantageous for compound A.

Suitable fillers are polyols such as mannitol, especially in granulated form, or else cellulose derivatives such as microcrystalline cellulose.

Suitable press additives are stearates, more particularly magnesium stearate.

Suitable disintegrants are cellulose derivatives, more particularly croscarmellose.

The active ingredient concentration can be from 0.1 mg/tablet to 10 mg/tablet, preferably from 0.37 mg/tablet to 8 mg/tablet, particularly preferably from 0.4 mg/tablet to 6 mg/tablet and extremely preferably from 0.5 mg/tablet to 5 mg/tablet.

The concentration 5 mg/tablet is given as an example.

Compound A is preferably in micronized form before and for the formulation into a dosage form.

Parenteral administration can be effected by avoiding an absorption step (e.g. intravenously, intraarterially, intracardially, intraspinally or intralumbally) or by involving absorption (e.g. intramuscularly, subcutaneously, intracutaneously, percutaneously or intraperitoneally). Suitable administration forms for parenteral administration are, inter alia, injection and infusion preparations in the form of solutions, suspensions, emulsions, lyophilisates or sterile powders.

Suitable for other routes of administration are, for example, inhalational dosage forms (including dry powder inhalers, nebulizers), nasal drops, nasal solutions, nasal sprays; tablets to be administered lingually, sublingually or buccally, films/wafers or capsules, suppositories, ear or eye preparations, vaginal capsules, aqueous suspensions (lotions, shake mixtures), lipophilic suspensions, ointments, creams, transdermal therapeutic systems (for example, patches), milk, pastes, foams, loose powders, implants or stents.

Compound A can be converted into the mentioned administration forms. This can be achieved in a manner known per se through mixing with inert, non-toxic, pharmaceutically suitable excipients. Said excipients include carriers (for example, microcrystalline cellulose, lactose, mannitol), solvents (e.g. liquid polyethylene glycols), emulsifiers and dispersants or wetting agents (for example, sodium dodecyl sulphate, polyoxysorbitan oleate), binders (for example, polyvinylpyrrolidone), synthetic and natural polymers (for example, albumin), stabilizers (e.g. antioxidants such as, for example, ascorbic acid), dyes (e.g. inorganic pigments such as, for example, iron oxides) and flavour and/or odour correctants.

The present invention further provides drugs containing compound A, typically together with one or more inert, non-toxic, pharmaceutically suitable excipients, and also for the use thereof for the above-mentioned purposes.

Compound A is formulated in a manner known per se to give pharmaceutical preparations by converting the active ingredient(s) into the desired administration form with pharmaceutically customary excipients.

Excipients which can be used in this connection are, for example, carrier substances, fillers, disintegrants, binders, humectants, lubricants, absorbents and adsorbents, diluents, solvents, cosolvents, emulsifiers, solubilizers, flavour correctants, colorants, preservatives, stabilizers, wetting agents, salts for altering the osmotic pressure or buffer.

Reference should be made here to Remington's Pharmaceutical Science, 15th ed. Mack Publishing Company, East Pennsylvania (1980).

The pharmaceutical formulations can be present
in solid form, for example as tablets, dragées, pills, suppositories, capsules, transdermal systems, or
in semi-solid form, for example as ointments, creams, gels, suppositories, emulsions, or
in liquid form, for example as solutions, tinctures, suspensions or emulsions.

Excipients in the context of the invention can be, for example, salts, saccharides (mono-, di-, tri-, oligo- and/or polysaccharides), proteins, amino acids, peptides, fats, waxes, oils, hydrocarbons and also derivatives thereof, it being possible for the excipients to be of natural origin or to be obtained synthetically or semi-synthetically.

Possibilities for oral or peroral administration are, in particular, tablets, dragées, capsules, pills, powders, granules, lozenges, suspensions, emulsions or solutions.

Possibilities for parenteral administration are, in particular, suspensions, emulsions and especially solutions.

The present invention provides for the use of compound A, more particularly compound A′, for treating lymphomas, more particularly diffuse large B-cell lymphomas or mantle cell lymphomas, rhabdomyosarcomas, neuroblastomas or squamous-cell carcinomas, especially of the lungs, the head and nape or the cervix.

Dosage and Treatment Regimen:

The dosage and the treatment regimen can and must be varied depending on the carcinoma type and the treatment goal.

The daily dose is generally between 0.5 mg and 20 mg and can be divided into a plurality of identical or different dosage units, preferably 2.

The preferred daily dose is between 1.0 mg and 15 mg and can be divided into a plurality of identical or different dosage units, preferably 2.

This applies both to monotherapy and to combination therapy with other anti-hyperproliferative, cytostatic or cytotoxic substances, the combination therapy possibly requiring a reduction in dose.

The treatment can be carried out for 2 to 60 days, the treatment preferably being followed by a treatment break of 2 to 30 days.

Treatment is successful when there is at least disease stabilization and the adverse effects occur to an extent which is easily treatable, but at least easily acceptable.

Compound A can be used on its own or, if required, in combination with one or more other pharmacologically effective substances, provided said combination does not lead to undesired and unacceptable adverse effects. The present invention therefore further provides drugs containing at least one of the compounds according to the invention and one or more further active ingredients, in particular for treating and/or preventing the above-mentioned diseases.

For example, compound A can be combined with known anti-hyperproliferative, cytostatic or cytotoxic substances for treating cancers. The combination of the compounds according to the invention with other substances in use for cancer therapy or else with radiotherapy is especially advisable.

Examples of suitable active ingredients for combination purposes include: abraxane, afinitor, aldesleukin, alendronic acid, alfaferone, alitretinoin, allopurinol, aloprim, aloxi, altretamine, aminoglutethimide, amifostine, amrubicin, amsacrine, anastrozole, anzemet, aranesp, arglabin, arsenic trioxide, aromasin, 5-azacytidine, azathioprine, BCG or tice-BCG, bestatin, betamethasone acetate, betamethasone sodium phosphate, bexarotene, bleomycin sulphate, broxuridine, bortezomib, busulfan, calcitonin, campath, capecitabine, carboplatin, casodex, cefesone, celmoleukin, cerubidine, chlorambucil, cisplatin, cladribine, clodronic acid, cyclophosphamide, cytarabine, dacarbazine, dactinomycin, daunoxome, decadron, decadron phosphate, delestrogen, denileukin diftitox, depo-medrol, deslorelin, dexrazoxane, diethylstilbestrol, diflucan, docetaxel, doxifluridine, doxorubicin, dronabinol, DW-166HC, eligard, elitek, ellence, emend, epirubicin, epoetin alfa, epogen, eptaplatin, ergamisol, estrace, estradiol, estramustine sodium phosphate, ethinyl estradiol, ethyol, etidronic acid, etopophos, etoposide, fadrozole, fareston, filgrastim, finasteride, fligrastim, floxuridine, fluconazole, fludarabine, 5-fluorodeoxyuridine monophosphate, 5-fluorouracil (5-FU), fluoxymesterone, flutamide, formestane, fosteabine, fotemustine, fulvestrant, gammagard, gemcitabine, gemtuzumab, gleevec, gliadel, goserelin, granisetron hydrochloride, histrelin, hycamtin, hydrocortone, erythro-hydroxynonyladenine, hydroxyurea, ibritumomab tiuxetan, idarubicin, ifosfamide, interferon alpha, interferon alpha 2, interferon alpha 2α, interferon alpha 2β, interferon alpha nl, interferon alpha n3, interferon beta, interferon gamma 1α, interleukin 2, intron A, iressa, irinotecan, kytril, lapatinib, lentinan sulphate, letrozole, leucovorin, leuprolide, leuprolide acetate, levamisole, levofolinic acid calcium salt, levothroid, levoxyl, lomustine, lonidamine, marinol, mechlorethamine, mecobalamin, medroxyprogesterone acetate, megestrol acetate, melphalan, menest, 6-mercaptopurine, mesna, methotrexate, metvix, miltefosine, minocycline, mitomycin C, mitotane, mitoxantrone, modrenal, myocet, nedaplatin, neulasta, neumega, neupogen, nilutamide, nolvadex, NSC-631570, OCT-43, octreotide, ondansetron hydrochloride, orapred, oxaliplatin, paclitaxel, pediapred, pegaspargase, pegasys, pentostatin, picibanil, pilocarpine hydrochloride, pirarubicin, plicamycin, porfimer sodium, prednimustine, prednisolone, prednisone, premarin, procarbazine, procrit, raltitrexed, RDEA119, rebif, rhenium-186 etidronate, rituximab, roferon-A, romurtide, salagen, sandostatin, sargramostim, semustine, sizofiran, sobuzoxane, solu-medrol, streptozocin, strontium-89 chloride, synthroid, tamoxifen, tamsulosin, tasonermin, tastolactone, taxotere, teceleukin, temozolomide, teniposide, testosterone propionate, tested, thioguanine, thiotepa, thyrotropin, tiludronic acid, topotecan, toremifene, tositumomab, trastuzumab, treosulfan, tretinoin, trexall, trimethylmelamine, trimetrexate, triptorelin acetate, triptorelin pamoate, UFT, uridine, valrubicin, vesnarinone, vinblastine, vincristine, vindesine, vinorelbine, virulizin, zinecard, zinostatin stimalamer, zofran; ABI-007, acolbifene, actimmune, affinitak, aminopterin, arzoxifene, asoprisnil, atamestane, atrasentan, BAY 43-9006 (sorafenib), avastin, CCI-779, CDC-501, celebrex, cetuximab, crisnatol, cyproterone acetate, decitabine, DN-101, doxorubicin MTC, dSLIM, dutasteride, edotecarin, eflornithine, exatecan, fenretinide, histamine dihydrochloride, histrelin hydrogel implant, holmium-166 DOTMP, ibandronic acid, interferon gamma, intron-PEG, ixabepilone, keyhole limpet hemocyanin, L-651582, lanreotide, lasofoxifene, libra, lonafarnib, miproxifen, minodronate, MS-209, liposomal MTP-PE, MX-6, nafarelin, nemorubicin, neovastat, nolatrexed, oblimersen, onco-TCS, osidem, paclitaxel polyglutamate, pamidronate disodium, PN-401, QS-21, quazepam, R-1549, raloxifene, ranpirnase, 13-cis-retinoic acid, satraplatin, seocalcitol, T-138067, tarceva, taxoprexin, thymosin alpha 1, tiazofurin, tipifarnib, tirapazamine, TLK-286, toremifene, transMID-107R, valspodar, vapreotide, vatalanib, verteporfin, vinflunine, Z-100, zoledronic acid, and also combinations thereof.

In a preferred embodiment, compound A of the present invention can be combined with the following active ingredients:

131I-chTNT, abarelix, abiraterone, aclarubicin, aldesleukin, alemtuzumab, alitretinoin, altretamine, aminoglutethimide, amrubicin, amsacrine, anastrozole, arglabin, arsenic trioxide, asparaginase, azacitidine, basiliximab, BAY 80-6946, belotecan, bendamustine, bevacizumab, bexarotene, bicalutamide, bisantrene, bleomycin, bortezomib, buserelin, busulfan, cabazitaxel, calcium folinate, calcium levofolinate, capecitabine, carboplatin, carmofur, carmustine, catumaxomab, celecoxib, celmoleukin, cetuximab, chlorambucil, chlormadinone, chlormethine, cisplatin, cladribine, clodronic acid, clofarabine, crisantaspase, cyclophosphamide, cyproterone, cytarabine, dacarbazine, dactinomycin, darbepoetin alfa, dasatinib, daunorubicin, decitabine, degarelix, denileukin diftitox, denosumab, deslorelin, dibrospidium chloride, docetaxel, doxifluridine, doxorubicin, doxorubicin+estrone, eculizumab, edrecolomab, elliptinium acetate, eltrombopag, endostatin, enocitabine, epirubicin, epitiostanol, epoetin alfa, epoetin beta, eptaplatin, eribulin, erlotinib, estradiol, estramustine, etoposide, everolimus, exemestane, fadrozole, filgrastim, fludarabine, fluorouracil, flutamide, formestane, fotemustine, fulvestrant, gallium nitrate, ganirelix, gefitinib, gemcitabine, gemtuzumab, glutoxim, goserelin, histamine dihydrochloride, histrelin, hydroxycarbamide, I-125 seeds, ibandronic acid, ibritumomab tiuxetan, idarubicin, ifosfamide, imatinib, imiquimod, improsulfan, interferon alpha, interferon beta, interferon gamma, ipilimumab, irinotecan, ixabepilone, lanreotide, lapatinib, lenalidomide, lenograstim, lentinan, letrozole, leuprorelin, levamisole, lisuride, lobaplatin, lomustine, lonidamine, masoprocol, medroxyprogesterone, megestrol, melphalan, mepitiostane, mercaptopurine, methotrexate, methoxsalen, methyl aminolevulinate, methyltestosterone, mifamurtide, miltefosine, miriplatin, mitobronitol, mitoguazone, mitolactol, mitomycin, mitotane, mitoxantrone, nedaplatin, nelarabine, nilotinib, nilutamide, nimotuzumab, nimustine, nitracrine, ofatumumab, omeprazole, oprelvekin, oxaliplatin, p53 gene therapy, paclitaxel, palifermin, palladium-103 seed, pamidronic acid, panitumumab, pazopanib, pegaspargase, PEG-epoetin beta (methoxy-PEG-epoetin beta), pegfilgrastim, peginterferon alfa 2b, pemetrexed, pentazocine, pentostatin, peplomycin, perfosfamide, picibanil, pirarubicin, plerixafor, plicamycin, poliglusam, polyestradiol phosphate, polysaccharide-K, porfimer sodium, pralatrexate, prednimustine, procarbazine, quinagolide, radium-223 chloride, raloxifene, raltitrexed, ranimustine, razoxane, refametinib, regorafenib, risedronic acid, rituximab, romidepsin, romiplostim, sargramostim, sipuleucel-T, sizofiran, sobuzoxane, sodium glycididazole, sorafenib, streptozocin, sunitinib, talaporfin, tamibarotene, tamoxifen, tasonermin, teceleukin, tegafur, tegafur+gimeracil+oteracil, temoporfin, temozolomide, temsirolimus, teniposide, testosterone, tetrofosmin, thalidomide, thiotepa, thymalfasin, tioguanine, tocilizumab, topotecan, toremifene, tositumomab, trabectedin, trastuzumab, treosulfan, tretinoin, trilostane, triptorelin, trofosfamide, tryptophan, ubenimex, valrubicin, vandetanib, vapreotide, vemurafenib, vinblastine, vincristine, vindesine, vinflunine, vinorelbine, vorinostat, vorozole, yttrium-90 glass microspheres, zinostatin, zinostatin stimalamer, zoledronic acid, zorubicin.

Promisingly, compound A can also be combined with biological therapeutics such as antibodies (e.g. avastin, rituxan, erbitux, herceptin, cetuximab) and recombinant proteins.

Compound A can also achieve positive effects in combination with other therapies directed against angiogenesis, such as, for example, with avastin, axitinib, regorafenib, recentin, sorafenib or sunitinib. Combinations with inhibitors of the proteasome and of mTOR and also antihormones and steroidal metabolic enzyme inhibitors are especially useful because of their favourable profile of adverse effects.

In general, the combination of compound A with other cytostatic or cytotoxic agents makes it possible to pursue the following goals:
improved efficacy in slowing the growth of a tumour, in reducing its size or even in completely eliminating it in comparison with treatment using an individual active ingredient;
the possibility of employing the chemotherapeutics used in a lower dosage than in the case of monotherapy;
the possibility of a more tolerable therapy with fewer adverse effects in comparison with individual administration;
the possibility of treating a broader spectrum of tumour diseases;
achieving a higher response rate to the therapy;
longer patient survival time in comparison with current standard therapy.

Furthermore, the compounds according to the invention can also be used in connection with radiotherapy and/or a surgical intervention.

### EXAMPLES

1. Preparation of the Compounds According to the Invention

The preparation of the compounds according to the invention is comprehensively described in PCT/EP2009/007247, the disclosure of which is referred to in the present application and which is incorporated into this application by reference.

A further developed preparation process is disclosed by PCT/EP2011/066295, the disclosure of which is likewise referred to in the present application and which is incorporated into this application by reference.

2. Proliferation Assay

Human tumour cells were originally obtained from the American Type Culture Collection (ATCC), the Deutsche Sammlung von Mikroorganismen and Zellkulturen (DSMZ) (German Collection of Microorganisms and Cell Cultures) in Braunschweig, CLS Cell Lines Service GmbH in Eppelheim, the Institut Gustave Roussy (Villejuif, France), or the Charité in Berlin (table 1). Adherently growing cells (A-673, RD, Rh30, Rh41, SK-N-AS, NCI-H2286, HCC-366, FaDu, CAL-33, RPMI-2650, SiHa) were plated out in a density of 3000-4000 cells/measurement point, depending on the rate of growth of the cell line, in a 96-well multititre plate in 200 μl of growth medium (DMEM/HAMS F12, 2 mM L-glutamine, 10% foetal calf serum). After 24 hours, the cells of one plate (zero plate) were dyed with crystal violet (see below), whereas the medium of the other plates was replaced with fresh culture medium (200 μl) to which the test substances were added in various concentrations (0 μM, and also in the range of 0.001-3 μM; the final concentration of the solvent dimethyl sulphoxide was 0.5%). The cells were incubated for 4 days in the presence of the test substances. Cell proliferation was determined by dyeing the cells with crystal violet: the cells were fixed at room temperature for 15 min by adding 20 μl/measurement point of an 11% strength glutaraldehyde solution. After washing the fixed cells three times with water, the plates were dried at room temperature. The cells were dyed by adding 100 μl/measurement point of a 0.1% strength crystal violet solution (pH adjusted to pH 3 by adding acetic acid). After washing the dyed cells three times with water, the plates were dried at room temperature. The dye was dissolved by adding 100 μl/measurement point of a 10% strength acetic acid solution. Absorbance was determined photometrically at a wavelength of 595 nm. The percentage change in cell growth was calculated by normalizing the measured values to the absorbance values of the zero plate (=0%) and the absorbance of the untreated (0 μM) cells (=100%). The measured data were normalized to 0% inhibition (cell proliferation without inhibitor) and 100% inhibition (zero plate). The IC₅₀ values were determined by means of a four parameter fit.

Cells growing in suspension (HBL-1, TMD-8, GRANTA-519, Jeko-1) were plated out in a cell density of 2000-4000 cells/measurement point, depending on the rate of growth of the cell line, in a black-walled, clear-bottom 96-well multititre plate in 100 μl of growth medium (DMEM/HAMS F12, 2 mM L-glutamine, 10% foetal calf serum). After 24 hours, cell density was determined in one plate (zero plate) by adding 60 μl/measurement point of CTG solution (Promega Cell Titer-Glo solution (catalogue numbers G755B and G756B)), subsequent incubation for 2 min followed by 10 min shaking (in the dark) and measurement of luminescence (VICTOR V, Perkin Elmer). For the test plates, the test substances were prepared in various concentrations (0 μM, and also in the range of 0.001-3 μM; the final concentration of the solvent dimethyl sulphoxide was 0.5%) as 3× concentrated solutions in fresh growth medium. Aliquots of 50 μl each were added to the cell suspensions and the cells were incubated for 4 days in the presence of the test substances. Subsequently, cell density was determined using CTG solution as described above and IC₅₀ values were calculated by means of a four parameter fit.

The cytotoxic action of BAY 1000394 on Jeko-1 and UPN-1 cells was also determined after 24 hours of incubation with the substance. To this end, 50 000 cells per measurement point were plated out in 96-well plates and incubated with BAY 1000394 in various concentrations in the range of 0.001 to 1.0 μM. After 24 hours, the viability of the cells was determined in accordance with the WST-1 method (Roche Diagnostics, catalogue number 11644807001).

The substances were investigated in the following cell lines, which, by way of example, represent the specified indications (table 1).

TABLE 1
List of the cell lines investigated and results of the proliferation assays.

| | Example | BAY 1000394 |
|---|---|---|
| Tumour indication | Cell line | IC₅₀ [mol/l] |
| Lymphoma | TMD-8 (Charité, Berlin) | 3.5E−08 |
| Diffuse large B-cell | HBL-1 (Charité, Berlin) | 2.4E−08 |
| lymphoma | | |
| Lymphoma | GRANTA-519 (DSMZ ACC-342) | 4.4E−08 |
| | Jeko-1 (DSMZ ACC-553) | 2.0E−08 |
| Mantle cell | UPN-1 (Institut Gustave Roussy) | 4.7E−09^{#} |
| lymphoma | Jeko-1 (Institut Gustave Roussy) | 3.1E−08^{#} |
| Rhabdomyosarcoma | A-673 (ATCC CRL-1598) | 9.8E−09 |
| | RD (CLS-300401) | 2.6E−08 |
| | Rh30 (DSMZ ACC-489) | 2.3E−08 |
| | Rh41 (DSMZ ACC-592) | 1.7E−08 |
| Neuroblastoma | SK-N-AS (ATCC CRL-2137) | 2.2E−08 |
| Squamous-cell | NCI-H2286 (ATCC CRL-5938) | 7.0E−09 |
| carcinoma, lung | HCC-366 (DSMZ ACC-492) | 2.0E−08 |
| Squamous-cell | FaDu (ATCC HTB-43) | 4.6E−08 |
| carcinoma, head | CAL-33 (DSMZ ACC-447) | 1.9E−08 |
| and nape | RPMI-2650 (DSMZ ACC-287) | 1.3E−08 |
| Squamous-cell | SiHa (ATCC HTB-35) | 1.0E−08 |
| carcinoma, cervix | | |

^{#}After 24 hours of incubation with the substance

The results of the proliferation assays demonstrate the efficacy of BAY1000394 in the human tumour cells investigated. These data suggest a possible use for BAY1000394 in the tumour types investigated.

1. A method of treating a tumour selected from lymphomas, rhabdomyosarcomas, neuroblastomas or squamous-cell carcinomas of the lungs, the head and nape or the cervix comprising administering to a patient in need thereof a therapeutically effective amount of (RS)-S-cyclopropyl-S-(4-{[4-{[(1R, 2R)-2-hydroxy-1-methylpropyl]oxy}-5-(trifluoromethyl)pyrimidin-2-yl]amino}phenyl)sulphoximide.

2. A method of treating a tumour selected from lymphomas, rhabdomyosarcomas, neuroblastomas or squamous-cell carcinomas of the lungs, the head and nape or the cervix comprising administering to a patient in need thereof a therapeutically effective amount of (R)-S-cyclopropyl-S-(4-{[4-{[(1R, 2R)-2-hydroxy-1-methylpropyl]oxy}-5-(trifluoromethyl)pyrimidin-2-yl]amino}phenyl)sulphoximide.

3. (canceled)

4. (canceled)

5. The method according to claim 1, wherein the tumour is a lymphoma.

6. The method according to claim 1, wherein the tumour is a diffuse large B-cell lymphoma or a mantle cell lymphoma.

7. The method according to claim 1, wherein the tumour is a neuroblastoma.

8. (canceled)

9. (canceled)

10. (canceled)

11. (canceled)

12. (canceled)

13. (canceled)

14. A method of treating a tumour selected from lymphomas, rhabdomyosarcomas, neuroblastomas or squamous-cell carcinomas of the lungs, the head and nape or the cervix comprising administering to a patient in need thereof a therapeutically effective amount of a pharmaceutical composition comprising (RS)-S-cyclopropyl-S-(4-{[4-{[(1R, 2R)-2-hydroxy-1-methylpropyl]oxy}-5-(trifluoromethyl)pyrimidin-2-yl]amino}phenyl)sulphoximide and at least one further active ingredient.

15. A method of treating a tumour selected from lymphomas, rhabdomyosarcomas, neuroblastomas or squamous-cell carcinomas of the lungs, the head and nape or the cervix comprising administering to a patient in need thereof a therapeutically effective amount of a pharmaceutical composition comprising (R)-S-cyclopropyl-S-(4-{[4-{[(1R, 2R)-2-hydroxy-1-methylpropyl]oxy}-5-(trifluoromethyl)pyrimidin-2-yl]amino}phenyl)sulphoximide and at least one further active ingredient.

16. The method according to claim 2, wherein the tumour is a lymphoma.

17. The method according to claim 2, wherein the tumour is a diffuse large B-cell lymphoma or a mantle cell lymphoma.

18. The method according to claim 2, wherein the tumour is a neuroblastoma.
